(19) United States
(12) Patent Application Publication
(10) Pub. No.: US 2012/0171728 A1
(43) Pub. Date: Jul. 5, 2012

(54) PROCESS FOR AMPLIFYING DNA USING TETRATETHYLENE GLYCOL, KIT OF PARTS THEREFOR AND USE THEREOF

(75) Inventors: Andy Wende, Hilden (DE); Hans Robert Attig, Hilden (DE); Ralf Himmelreich, Langenfeld (DE)

(73) Assignee: QIAGEN GmbH, Hilden (DE)

(21) Appl. No.: 13/380,002

(22) PCT Filed: Jun. 15, 2010
(86) PCT No.: PCT/EP2010/058364

§ 371 Date: Mar. 9, 2012

(30) Foreign Application Priority Data
Jun. 22, 2009 (EP) 09163338.8

(51) Int. Cl. C12P 19/34; C07C 43/11; C12N 9/12

(52) U.S. Cl. 435/91.2; 435/194; 568/623

(57) ABSTRACT

Disclosed is a process for amplifying DNA, a process for preparing and amplifying DNA, a kit of parts for DNA amplification and DNA preparation, and the use thereof, all of which are characterized by the use of tetraethylene glycol. Moreover, the use of tetraethylene glycol in a reaction solution by carrying out a DNA amplification and a reaction solution containing tetraethylene glycol for carrying out a DNA amplification are disclosed.

The invention relates to a process for amplifying DNA or a process for preparing and amplifying DNA, a kit of parts for DNA amplification, and the use thereof, a reaction solution for carrying out DNA amplification, and the use of tetraethylene glycol in a reaction solution for carrying out DNA amplification.

### BACKGROUND

It is increasingly important to detect bioparticles such as bacteria, viruses, fungi, protozoa or the like in samples from various origins.

One option for doing so is the release of the nucleic acids of the cells contained in a sample, their subsequent specific amplification, and then detection thereof. The following steps are required for this purpose:
Sampling of material (e.g. blood, sputum, urine, feces, liquor, as well as swab samples from the oral cavity, nasal region, genital tract; but also forensic samples, soil samples, etc.)
Lysis (disruption of the cells)
Nucleic acid preparation
Nucleic acid amplification
Detection of the amplified nucleic acids

This method is known in principle (e.g. Bowlen & Dürre, Nucleic Acids Isolation Methods, American Scientific Publishers, 2002).

In the known processes for preparing nucleic acids, ethanol is frequently used as a solvent. Today, ethanol is generally used, for example, as a washing buffer additive (e.g. DE-A 198 56 064, US 2005/0079535).

However, ethanol is classified as a hazardous material (HAZMAT, hazardous material) by IATA (International Air Transportation Association). Thus, additional fees and taxes apply in case of transport by plane. Moreover, complex steps for removing ethanol are required subsequent to the washing process because small quantities of ethanol may already strongly interfere with or completely block subsequent reactions such as PCR.

There are already efforts being taken to replace ethanol with other substances in processes for purifying nucleic acids.

For example, the earlier application EP 08163623.5 by the applicant proposes the use in the adsorption step of binding mediators, which are considered harmless according to the IATA guidelines. Among others, tetraethylene glycol is also mentioned as a binding mediator.

Another earlier application by the applicant, EP 08163624, proposes washing buffers that contain less than 24 vol %, particularly preferably no ethanol. Various solvents can be used as solvents; the washing buffer may, among other things, also contain tetraethylene glycol.

DE-A 102 53 351 points out that the use of alcohol or acetone in processes for purifying nucleic acids has great drawbacks. For example, even traces of alcohol affect the subsequent reactions to a very considerable extent. Therefore, customary purifying processes for nucleic acids always include an ethanol removal step. In order to avoid these issues, the document proposes avoiding the use of alcoholic components altogether. Among other things, an alcohol-free washing buffer is disclosed which contains NaCl, MgCl₂ as well as Tris/HCl; no mention is made of tetraethylene glycol.

WO 2004/042058 describes buffer formulations for the isolation, purification and recovery of short-chain nucleic acids that contains monovalent and multivalent cations. An alcohol-free washing buffer containing NaCl, MgCl₂ and Tris/HCl is also disclosed here.

WO 02/44400 discloses, as a reagent for extracting nucleic acids, an aqueous solution containing sodium metasilicate and a substituted ether. No mention is made of washing buffers.

U.S. Pat. No. 5,610,287 describes alcohol-free washing buffers containing Tris/HCl, NaCl as well as Tween 20.

WO 2008/071384 discloses the use of tetraethylene glycol dimethyl ether in various steps of DNA preparation and purification.

However, none of these methods have so far led to a substitute solution for ethanol that is satisfactory in every respect. In particular, it has not been cleared up, for any of the above-described substitute solvents, how the presence of the washing buffer constituents affects the subsequent DNA, amplification. For even small quantities of solvent usually affect the efficiency of PCR to a very considerable extent.

Therefore, the object of the invention is to provide an improvement of the preparation step, in particular in the preparation of DNA, in which the PCR carried out subsequently is not affected by the solvent used.

### DISCLOSURE OF THE INVENTION

Surprisingly, it was found that this object can be accomplished by using tetraethylene glycol, and that the presence of tetraethylene glycol does not affect the reactions steps carried out subsequent to the DNA preparation.

The invention therefore provides processes for amplifying DNA in which the reaction solution contains tetraethylene glycol.

In this case, the tetraethylene glycol in the reaction solution originates from the eluate of the DNA preparation on solid surfaces that was used for preparing the DNA.

Moreover, the invention relates to a process for the preparation and amplification of DNA, comprising the following steps:
a) immobilizing DNA contained in a solution on a surface
b) washing the surface with a washing buffer
c) recovering the DNA by elution,
d) carrying out a DNA amplification with the eluate obtained in step c), characterized in that the washing buffer comprises tetraethylene glycol.

Preferably in this case, the tetraethylene glycol quantity in the washing buffer is at least 10 vol %, preferably at least 48 vol %, and more, preferably at least 60 vol %.

Also preferably, the volume of the eluate in step d) is at least 10 vol %, preferably at least 30 vol %, more preferably at least 50 vol %, based on the entire reaction volume of the amplification reaction.

Furthermore preferred is a configuration of the process in which the tetraethylene glycol content in the entire reaction volume of the amplification is up to 20 vol %, preferably up to 12 vol %, more preferably up to 10 vol %.

Moreover, the washing buffer may contain one or more constituents selected from the group comprising chaotropic salts and buffering substances.

DNA amplification can be carried out as an end-point PCR, quantitative PCR or isothermal amplification.

Furthermore, the invention provides a process according to one or more of the preceding embodiments which is carried, out in a microfluidic device (lab on a chip, LoC).

Another aspect of the invention is a kit of parts for DNA amplification and DNA preparation according to the process of one or more of the above embodiments, comprising:
a solution 1 as a washing buffer containing tetraethylene glycol, and
reagents required for carrying out the DNA amplification.

Preferably, the kit of parts moreover comprises one or more of the following constituents, selected from
a solution 2, comprising a binding mediator,
a solution 3, comprising an elution agent,
a solution 4 comprising a lysis buffer and optionally one or more proteases;

a microfluidic device (lab on a chip, LoC).

The invention further relates to the use of the above-mentioned kit of parts for the preparation and amplification of DNA obtained from biological materials, preferably from samples taken for molecular-diagnostic purposes (human and veterinary medicine), in particular from forensic samples and from viruses or bacteria contained in biological samples, such as, for example, blood.

Finally, the invention relates to the use of tetraethylene glycol in a reaction solution for carrying out a DNA amplification, in particular an end-point PCR, a quantitative PCR or an isothermal amplification; as well as a reaction solution for carrying out a DNA amplification, comprising reagents required for amplification and tetraethylene glycol, wherein the tetraethylene glycol content in the reaction solution is up to 20 vol %, preferably up to 12 vol %, more preferably up to 10 vol %.

### DESCRIPTION OF THE FIGURES

FIG. 1 shows the result of the PCR carried out in Example 1, which was carried out with genomic DNA from E. coli O157:H7 in whole blood or PBS with ethanol as a washing buffer additive.

FIG. 2 shows the result of the PCR with genomic DNA from E. coli O157:H7 in PBS with ethanol and tetraethylene glycol, respectively, as a washing buffer additive.

FIG. 3 is the photograph of an agarose gel, which shows the result of an end-point PCR of purified human gDNA in the presence of rising tetraethylene glycol concentrations in the reaction batch.

FIGS. 4 and 5 show the result of amplifications of RNA from the bacteriophage fr in blood carried out in the presence of ethanol and tetraethylene glycol, respectively.

FIG. 6 shows the result of an qPCR with purified RNA from the bacteriophage fr and purified human gDNA, respectively, in the presence of rising tetraethylene glycol concentrations in the reaction batch.

According to the invention, DNA means genomic DNA, gDAN, plasmid DNA or artificially produced DNA, such as, for example, short DNA fragments. It can be obtained from any cell or virus or be artificially produced. Preferably, the DNA has a length of 12 or more base pairs. It is obtained, for example, from prokaryotic or eukaryotic cells, such as eubacteria (e.g. gram-positive or gram-negative bacteria), archaea, pathogenic bacteria, eukaryotic microorganisms or cells of a multi-cellular organism, e.g. blood cells, tissue cells, etc. It may originate from a natural, artificially modified or artificially produced virus, e.g. a virulent, attenuated or non-infectious virus. It may also originate from fungi, yeasts, parasites, protozoa or other microorganisms.

Those microorganisms or cells from which DNA is obtained according to the invention can be obtained from any sample, e.g. samples from humans, animals, plants or soil samples, e.g. blood, urine, tissue samples, hair, saliva, stool, serum, cerebrospinal fluid, forensic samples, soil samples, parts of plants such as stalks, leaves, seeds, blossoms or roots.

The process according to the invention is particularly advantageous for amplifying DNA from cells present in the sample in very small quantities, such as, for example, DNA from viruses present in low titers in whole blood, or DNA from forensic samples.

It was found that the process according to the invention is not suitable for the preparation of RNA. This reason for this is probably that the reverse transcriptase required for the transcription of the eluted RNA into DNA is considerably more susceptible to tetraethylene glycol than to the ethanol usually used as a washing buffer additive.

In its most general embodiment, the invention relates to carrying out a DNA amplification in particular by means of PCR or isothermal amplification, with tetraethylene glycol being contained in the reaction solution. This preferably stems from the washing buffer used in the preceding DNA preparation, i.e. from the eluate obtained subsequent to the DNA preparation. However, it may also be added to the reaction solution not until later. It was found that even larger quantities of tetraethylene glycol do not inhibit the PCR, which is very surprising in view of the fact that the usually used ethanol already leads to considerable interference in the PCR in very small quantities, and that, as experiments by the applicant have shown, even small quantities of triethylene glycol (4 vol % of the reaction solution for PCR) already lead to a complete inhibition of the PCR.

According to the invention, the reaction solution used for carrying out the DNA amplification can contain up to 20 vol % tetraethylene glycol, more preferably up to 12 vol % and still more preferably up to 10 vol % tetraethylene glycol. It was found that no inhibition of the PCR takes place even in case such large quantities tetraethylene glycol are present.

In principle, the preparation of the DNA may take place in any known way.

Preferably, the procedure according to the invention is as follows:

The first thing to take place is the lysis of the cells, i.e. the disruption and release of the DNA contained therein in the known manner by heating, ultrasound or grinding by means of beads, e.g. glass beads. Lysis takes place in a conventional lysis buffer. The lysis buffer is, for example, the buffer AL (QIAGEN GmbH; Hilden, Germany). Other lysis buffers are known to persons skilled in the art and are selected to be suitable for the microorganism to by lysed, as are the suitable lysis conditions. Several different lysis buffers may also be used. For example, PBS buffer (phosphate-buffered saline solution) containing proteinase K and lysozyme may also be used as a buffer. Optionally, an incubation process is carried out first and then another buffer is optionally added, such as, for example buffer G (GITC (guanidinium thiocyanate)/Nonidet® (nonylphenylethylene glycol)) and an incubation is carried out again. The suitable volume of the buffer(s) must be determined depending on the sample material and is, depending on the sample material, between 50 μl and 1 ml, for instance. The temperature and duration for the incubation is in each case suitably selected. Since some lysis processes are carried out by using enzymes, the selected temperature also depends on the activity of the enzymes. Zymolase, lyticase and lysozyme are used, as a rule, in a temperature range of 20° C.-37° C. Incubation with proteases, e.g. proteinase K or subtilisin, is usually carried out of 50-60° C. The duration of incubation is, as a rule, 5 to 20 minutes, preferably about 10 minutes. Incubation can be carried out, for example, in a thermomixer by means of shaking, e.g. at about 1400 r.p.m., or also on a Vortex® device (maximum level)), optionally with the addition of glass beads.

Subsequent to the lysis, the isolation of the nucleic acids released by lysis takes place by means of immobilization (adsorption) on solid surfaces.

In the purifying processes using adsorption, the selective binding, of the constituents of the contents of the lysate to or on a solid supporting or carrier material (“binding”, immobilizing, step a)), the removal of undesired constituents from the solid supporting or carrier material (“washing”, step b)), and the elution of the desired constituent (“elution”, step c)). This process is also referred to as “bind-wash-elute”.

The surfaces on which the DNA is immobilized according to the invention are conventional surfaces known in the prior art, such as membranes, plates or beads. They consist of a plastic material such as, for example polystyrene, mineral material, such as glass powder, diatomaceous earth, silica gels, magnetic particles, non-woven glass-fibers, diaphragm filters, metal oxides, latex particles. Silica-based particles are most frequently used. Magnetic silica particles provided in the MagAttract®-Kit (Qiagen. GmbH, Hilden, Germany) can be used particularly advantageously. The DNA is, immobilized in such a way that it is not detached from the carrier in any appreciable amounts by washing with water.

Usually, a washing step with a washing buffer follows the immobilization in order to remove undesired lysates also bound to the surface.

Generally, washing buffers are aqueous solutions. According to the invention, the washing buffers preferably contain tetraethylene glycol. Preferably, the tetraethylene glycol content in the washing buffer is at least 10 vol %, more preferably at least 48 vol %, and most preferred at least 60 vol %. Furthermore, the washing buffer comprises substances that are usually used for washing buffers, in particular (purified) water, salts such as, for example, guanidinium hydrochloride, sodium chloride, and buffers such as Tris/HCl. Particularly preferably, ethanol can be replaced with tetraethylene glycol in the known washing buffers AW1 and AW2 by the company Gingen, so that particularly preferred washing buffers according to the invention are TEG1 (48 vol % tetraethylene glycol, 2 M guanidinium hydrochloride) and TEG2 (60 vol % tetraethylene glycol, 100 mM NaCl, 10 mM Tris/HCl pH 7.5).

The pH value is preferably set to a desired value using conventional buffering substances. Buffering substances include, for example, Tris/HCl, sodium acetate, maleate, sodium citrate. The pH of the washing buffer used according to the invention is preferably 7 to 9.

Possible chaotropic salts that can be added to the washing buffer include, according to the invention, in particular barium salts, thiocyanates, perchlorates, guandinium salts and in particular sodium chloride, guandinium hydrochloride, sodium iodide, sodium perchlorate and guandinium thiocyanate.

After washing, a drying process may optionally be carried out, if required (e.g. drying in an open vessel in air, “air dry”), or a washing process may be carried out (“water rinse”). In the process, water is conducted over the solid-phase particles without homogenizing them in the process. This process reduces the quantity of binding mediator (e.g. ethanol) present without detaching the bound nucleic acid from the solid phase. Generally, however, this is not necessary if tetraethylene glycol is used in the washing buffer because it does not interfere with the subsequent reactions. Drying and/or washing with water is necessary for removing ethanol only in the case where the washing buffer contains no tetraethylene glycol but ethanol, i.e. if tetraethylene glycol is only added to the PCR reaction mixture.

Elution is subsequently carried out in order to detach the nucleic acid from the surface again. As a rule, this is done using a weakly saline, optionally buffered, aqueous, pH-stabilized solution. Suitable elution solutions are known to the person skilled in the art.

The DNA preparation thus obtained is then amplified, e.g. by PCR or isothermal amplification.

The preparation of DNA with the so-called MagAttract® technology, in which the DNA is adsorbed on magnetic silica particles (MagBeads), is particularly preferred according to the invention. These particles are available from Qiagen. The reason for this is that, when using these particles, substances from the washing buffer, such as, for example, ethanol or tetraethylene glycol, can be removed only with difficulty. This can be done successfully only to a partial extent by drying (air dry), and slightly better by washing. However, both processes mean complex additional steps in the purification protocol. However, residual ethanol present in the eluate interferes with the subsequent PCR to a considerable extent up to and including total failure, whereas, surprisingly, tetraethylene glycol does not interfere with it.

The PCR technique is comprehensively described in, for example, PCR Technology: Principles and Applications for DNA Amplificaition, Erlich, editor (1992); PCR Protocols: A Guide to Methods and Applications, Innis et al., Hrsg. (1990), R. K. Saiki et al., Science 230:1350 (1985) and U.S. Pat. No. 4,683,202.

PCR is an enzymatic method for the synthesis of specific nucleic acid sequences using two oligonucleotide primers (probes) that hybridize with opposite strands and flank a section of a target nucleic acid that is of interest. A series of repeated reaction steps comprising template denaturation, primer annealing and extension of the hybridized primer by DNA polymerase results in the exponential accumulation of the specific target fragment, whose ends are defined by the 5′-ends of the primer. The PCR method uses repeated cycles of DNA synthesis for the replication of the target nucleic acid. In the basic embodiment, PCR comprises the following steps:
Adding specific primers to a sample which is assumed to contain the target nucleic acid. The primers are selected in such a way that they bind to complementary DNA strands present in opposite strands of the DNA. The target nucleic acid is localized within the primer binding sites and is a marker of the organism to be detected.
Adding the four desoxynucleoside triphosphates, magnesium sulfate-containing buffer, polymerase enzymes and differrent additives and cosolvents, mixing with the sample and the primers
Denaturing the sample by heating, separation of the DNA into two complementary strands
Cooling, thus binding (hybridization) of the primers to the respective binding sites on the complementary DNA strands
Primer extension on the DNA template by DNA polymerase.

These three steps, denaturing, hybridization and extension are repeated 40 to 50 times and result in an exponential amplification of the target sequences.

According to the invention, the PCR in step d) can be carried out as an end-point PCR or as a realtime PCR (quantitative PCR, qPCR). The procedure is known to the person skilled in the art. EP-A-0 512 34, EP-A-0 640 828, EP-A-0 519 338 (F. Hoffmann-La Roche AG) disclose, for example, qPCR. For this purpose, systems are on offer commercially, e.g. TadMan® (Roche Molecular Systems, Inc. Branchburg Township, N.J.).

According to the invention, all reagents customary for DNA PCR and kits available therefor can be used for carrying out the PCR. The primers, desoxynukleotide triphosphates, polymerases, the additives, cosolvents and marker probes are, as was described in the introduction, known to the person skilled in the art and are suitably selected. For example, appropriate PCR reagent sets by QIAGEN, such as QuantiTect® Probe PCR Mastermix or QuantiFast® Probe RT-PCR MasterMix, can be used for a TacqMan® analysis. For example, QuantiTect® PCR-Mastermix, QuantiTect®5× Virus-Mastermix or HotStarTaq®-Mastermix is, according to the invention, suitable for carrying out the PCR.

The primers are suitably selected and can be synthetically produced with methods known to persons skilled in the art, or are commercially available, for example from the companies Operon or MWG Biotech.

All markers known to persons skilled in the art can be used as markers. In particular, fluorescent markers such as 6-FAM, TET, Quasar® and/or HEX, are used for detection. Depending on the requirements, the person skilled in the art will also use other fluorophores.

Water is usually used as a solvent for the PCR reagents and primers.

According to the invention, the reaction solution for amplification contains tetraethylene glycol, preferably in an amount of up to 20 vol %, preferably up to 12 vol %, more preferably up to 10 vol %, based on the reaction solution. Surprisingly, it was found that the PCR proceeds very reliably and can be carried out successfully even in the presence of such large quantities of tetraethylene glycol.

In contrast, a reaction solution that contains already small amounts of tetraethylene glycol (e.g, from 1 vol %) is unsuitable for the amplification of RNA because the amplification efficiency deteriorates extremely in this case. Most frequently, no reaction can be observed anymore during RNA amplification at a content of 9 vol % TEG in the reaction batch.

The temperature cycles of the PCR are selected by the person skilled in the art in accordance with the PCR chemistry used, the underlying initial DNA and the primer selected and can be, for example, 15 min at 95° C. and 40× (15 seconds at 95° C., 1 minute at 60° C.). Temperature monitoring and the heating and cooling processes take place in the usual manner.

According to the invention, the DNA amplification can also be carried out as an isothermal amplification. Isothermal amplification processes are known in the prior art. They have in common that no temperature cycles are being used but that a certain reaction temperature is continually maintained. Examples for such processes that are suitable for DNA amplification and which therefore may be used according to the invention, include HDA (Helicase Dependent Amplification, see M. Vincent et al., EMBO, reports 2004, 795-800), RPA (Recombinase Polymerase Amplification, see O. Piepenburg et al., PloS Biology, 2006, 1115-1120), EXPAR (exponential amplification reaction, see e.g. J. Van Ness et al., PNAS 2002, 4504-4509].

A Hugl-PCR may also be carried out. Hugl is a target in human genomic DNA. The primer pair used for this PCR was developed by Qiagen some time ago. The PCR carried out therewith is very susceptible to interferences and is therefore used for demonstrating that preparations produced by DNA are particularly clean or contain no or almost no inhibitory contaminations.

The detection of the amplification products can take place in different ways, as was described in the introduction, depending on the markers used. This is known to the person skilled in the art. If fluorescent markers are used, detection is carried out with fluorescence spectrometry. In conventional PCR processes, up to 6 different fluorophores can be detected simultaneously (6 channel multiplexing). Then, the number of PCR cycles from which a significant drop of the fluorescence of the specific marker occurs (Ct, threshold cycle) is determined.

The classic PCR reaction is an end-point PCR. It is thus possible to detect the presence or absence of a target DNA; however, the method does not yield any information as to the initial concentration of the target DNA.

Real-Time PCR(RT-PCR; realtime polymerase chain reaction; also referred to as quantitative PCR (qPCR)) was developed in recent times. It enables the simultaneous observation of several nucleic acid amplification reactions by PCR, with the accumulated data being used for the quantitative determination of the initial concentration of a target nucleic acid. Therefore, this PCR reaction is also referred to as multiplex PCR. Real-Time PCR is disclosed, for example, in EP-A-0 512 34, EP-A-0 640 828, EP-A-0 519 338 (F. Hoffmann-La Roche AG). For this purpose, systems are on offer commercially, e.g. TadMan® (Roche Molecular Systems, Inc., Branchburg Township, N.J.). The method enables the rapid and precise quantification of the initial number of copies over a very broad concentration range. It is suitable for many applications such as gene expression studies, sequence and mutation analyses, testing of virus titers, the detection of pathogenic or genetically modified organisms. A Real-Time PCR kit with sequence-specific probes is available, for example, from Eppendorf AG, Hamburg (RealMasterMix Probe®) or from Qiagen GmbH, Hamburg (Artus® PCR Kits).

In the TagMan® PCR, the oligonucleotide probes are designed in such a way that they bind to the target nucleic acid sequence between the extension primers. Each oligonucleotide probe is marked on the 5′ end with a reporter molecule, such as a fluorophore, and on the 3′ end with a reporter molecule quencher. The marked probes are added to the PCR reaction mixture together with the primers and the sample. After denaturing, the reaction mixture is cooled off, wherein the marked probes preferably bind to the target nucleic acid sequence. Then, a cooling process is carried out down to the optimum temperature for primer binding and extension. During polymerization, when the DNA polymerase proceeds along the target nucleic acid strand from the 3′ end to the 5′ end with nucleotides being bound to the growing primer, the primer encounters the 5′ ends of the marked probes previously bound to the target nucleic acid strand. When the DNA polymerase encounters these marked probes, it exercises its 5′-3′-exonuclease activity and disintegrates the marked probes. Fluorophores and quenchers are released into the reaction mixture in the process. The TagMan® assay is designed in such a way that reporter molecules present within a predetermined vicinity of the quencher molecule emit almost no fluorescence. Therefore, no fluorescence due to the reporter molecule is initially detected in the PCR mixture, because the fluorescence of the reporter molecule is quenched by the quencher molecule located in spatial proximity. During PCR, the 5′ fluorescence-marked probes are released and separated in the process from the 3′ fluorescence quenchers. After the release, the fluorescent marker is quenched no longer and can therefore be detected by fluorescence spectrometry or other suitable means. Non-bound probes present in the reaction mixture are not an interference because they remain quenched. Marked probes which are unspecifically bound to nucleic acid sequence that have nothing to do with the target nucleic acid, also do not interfere because they are bound and therefore remain quenched. Therefore, every free reporter molecule detected in the reaction mixture is directly proportional to the quantity of the originally specifically bound marked probe, and therefore of the target nucleic acid. For detecting different target nucleic acids in a sample, probes with different fluorescence reporter molecules are used which fluoresce in different wavelength ranges and therefore enable the simultaneous detection of different target DNAs. 6-FAM, VIC, Bodipy-TMR, JOE and HEX are known and suitable as fluorescence reporters, TAMRA, MBG, Black Hole Quencher 1 (BHQ1), Black Hole Quencher 2 (BHQ2), Black Hole Quencher 3 (BHQ3), BodyPi 564/570 and Cy5 or the like are known and suitable as quenchers. The person skilled in the art will suitably select the reporters and quenchers preferably in such a way that a mutual superposition of the fluorescent emissions (“bleed through”) is avoided. The combination 6-FAM/BHQ1, for example, is particularly suitable.

Apart from fluorescent markers, radioactive markers, chemiluminescent markers, paramagnetic markers, enzymes and enzyme substrates may also be used as markers.

Subsequent to amplification, the detection of the nucleic acids takes place by means of fluorescence spectrometry, as described above, or if other markers are used, by means of other detection methods.

The aforementioned nucleic acid preparation and amplification and detection methods can be carried out on a laboratory scale, i.e. on the micron or millimeter scale. Alternatively, microfluidic processes were developed in recent times. These may also be used according to the invention.

Microfluidics is understood to be handling and working with small quantities of liquid that have a volume that is lower by powers of ten than normal drops of liquid.

Devices for carrying out microfluidic methods that process a sample until the desired measurement result is obtained are also referred to as lab on a chip (LoC), i.e. disposable material as a laboratory on a chip of the approximate size of a credit card, with which molecular-biological determinations can be carried out, e.g. the determination of pathogens of an infection, foodstuff control, veterinary diagnostics, biological weapons analysis, environmental analysis. Analysis requires only few personnel and no special education or training for operation. LoC are inserted into an operator instrument having the size of a video cassette recorder, which then outputs the result. An LoC combines on a single chip microfluidic functions for sample extraction and enrichment of the analyte, for signal amplification and detection. The operation of the LoC and the operating unit is very simple, and analysis is extremely rapid compared with conventional analysis methods (30-60 minutes total process time, compared with at least 6 hours).

### EXAMPLES

Escherichla coli O157:H7 is available from the American Type Culture Collection under No. ATCC 700728.
Purified human gDNA was produced from human blood using the QIAamp® DNA Midi Kit.
Bacteriophage fr is available from the American Type Culture Collection under No. ATCC 15767-B1.

### Abbreviations Used Below:

QA=QIAamp®, available from Qiagen
MasG=MagAttract® Suspension G, available from Qiagen
PBS=Phosphate buffered saline (solution)
AW=QIAGEN washing buffer AW (1/2), available from Qiagen
TEG=Tetraethylene glycol
SPE=Nucleic acid solid phase extraction (by means of QA o. MasG)
qPCR=Quantitative PCR
RT-qPCR=Reverse transcription-quantitative PCR
BHQ=Black hole quencher

The effects of various washing procedures on the efficiency of end-point and realtime PCR, respectively, are described in the following examples. The washing process with TEG described herein was respectively carried out with the buffers TEG1 and TEG2 as a substitute from AW1 and AW2. The buffers TEG1 and TEG2 are composed as follows:

TEG1: 48 vol % tetraethylene glycol, 2.5M guanidinium-HCl, remainder water
TEG2: 60 vol % tetraethylene glycol, 100 mM NaCl, 10 mM Tris/HCl pH 7.5, remainder water

### Example 1

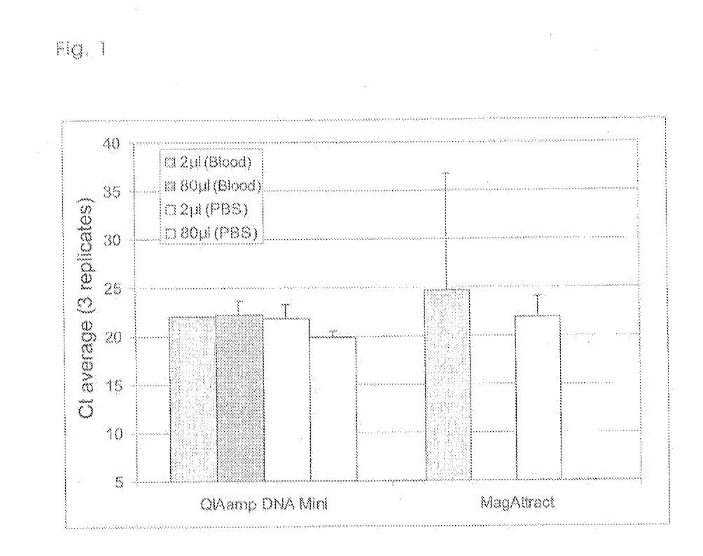

40 μl of an overnight culture of E. coli O157:H7 was added to 800 μl whole blood or PBS, respectively. To this, 80 μl QIAGEN proteinase K and 800 μl buffer AL were added and this was mixed. This mixture was now incubated for 15 at 56° C. The lysate thus produced was used for all further purifications of the DNA obtained, DNA purification took place three times in each case, using the QIAamp® DNA Mini Kit or by means of a manual MagAttract® protocol, respectively. Purification using a QA DNA Mini Kit® was carried out in accordance with the instructions by the manufacturer and comprised 2 washing steps with AW1 and AW2, respectively, a short drying of the bound nucleic acid by air and elution with deionized water. The MasG® protocol comprised the following steps: 200 μl lysate was mixed with 200 μl isopropanol and 20 μl MasG® by means of short vortexing and then shaken for 5 min at 1400 r.p.m. on a thermomixer. Then, the MasG® beads were magnetically separated and the supernatant was removed and discarded. The MasG® beads were washed by iterative resuspension in the corresponding buffer, subsequent magnetic separation and removal of the supernatant four times with AW1 and AW2, respectively, in each case. Then, the separated beds were dried in air for 15 min in an open 1.5 ml reaction vessel and then resuspended in 100 μl water. For elution of the DNA, the beads distributed in the water were shaken for 2 min. Then, the beads were separated and the supernatant containing the DNA was removed. The DNA solutions thus obtained were added to a qPCR reaction. This was carried out by receiving 40 μl QuantiTect® PCR-Mastermix (Qiagen GmbH), 40 pmol forward primer (SEQ ID NO:1; 5′-CGATGATGCTACCCCTGAAAAACT-3′), 40 pmol reverse primer (SEQ ID NO:2; 5′TATTGTCGCTTGAACTGATTTCCTC-3′). 20 pmol probe oligo (SEQ ID NO:3; FAM-CGTTGTTAAGTCAATGGAAAACCTG-BHQ1-) in a 96-well PCR plate and drying the mixture overnight at 37° C. The dried-up reaction mixture was rehydrated with 80 μl eluate or 78 μl water plus 2 μl eluate, respectively, and shaken for 5 min at 1400 r.p.m. on a thermomixer. Then, the redissolved reaction mixture was subjected to the following temperature profile in a real-time cycler (MJ Research Opticon, Biorad): 15 min 95° C., 40×[15 sec 95° C., 1 min 60° C.]. The result is shown in FIG. 1.

Result: While QA preparation permits the use of large quantities of eluate for the subsequent preparation, this is not possible for eluates obtained with the MasG® technology. One possible reason for this is the incomplete removal of ethanol from the MasG® beads prior to DNA elution.

### Example 2

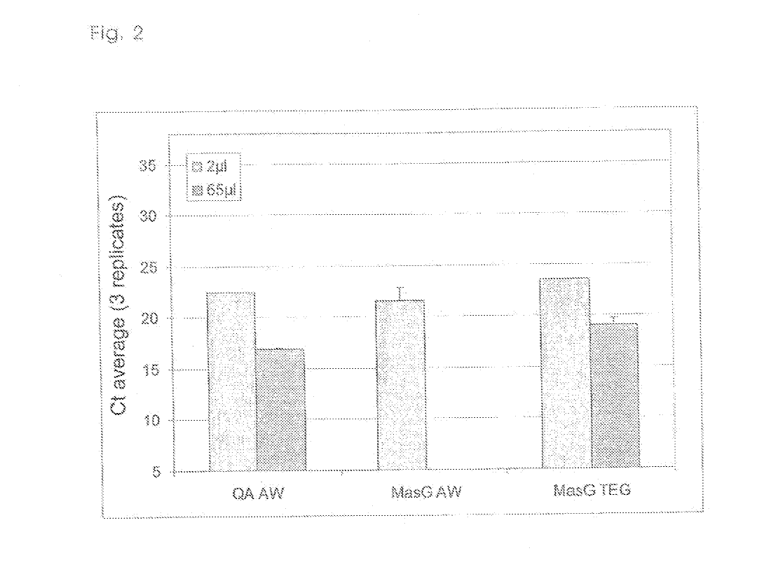

As described in Example 1, a joint lysate of a mixture of 100 μl E. coli O157:H7, 3-ml PBS, 300 μl Qiagen proteinase K and 3 ml GIN3 [3M guanidinium isothiocyanate, 20% NP-40 (Nonidet P-40, octylphenoxypolyethoxyethanol)]. This joint lysate was carried out exactly as in Example 1, with the following exceptions. For washing the bound nucleic acid, TEG1/TEG2 was also used apart from AW1/AW2. Washing during the MasG®-based purification was carried out twice in each case with washing buffer 1 and 2. In the case of the samples treated with TEG-based washing buffer, the DNA-loaded MasG® beads were rinsed with 500 μl instead of being dried for 15 minutes. During this rinsing process, the beads were not resuspended, but the reaction vessel remained in the magnetic holder during the rinsing process, the holder ensuring that the beads were separated from the liquid as a compact pile. This step is necessary because TEG, in contrast to ethanol, virtually does not evaporate at room temperature due to its very high boiling point. The DNA solutions thus obtained were added to a qPCR reaction. This took place exactly as described in Example 1, by rehydration of a previously dried reaction mix with 65 μl eluate, or 63 μl water pülaus 2 μl eluate, respectively. The PCR conditions are also identical to those in Example 1. The result is shown in FIG. 2.

Result: If large quantities of eluate from a MasG® DNA preparation are to be added to a subsequent qPCR, the use of ethanol-containing washing buffers (AW1/2) leads to inhibition effects that cause a total failure of the PCR. If TEG-containing washing buffers are used instead (TEG1/2), a previously dried PCR mastermix may be reconsitituted exclusively with this eluate and a qPCR can be carried out successfully.

### Example 3

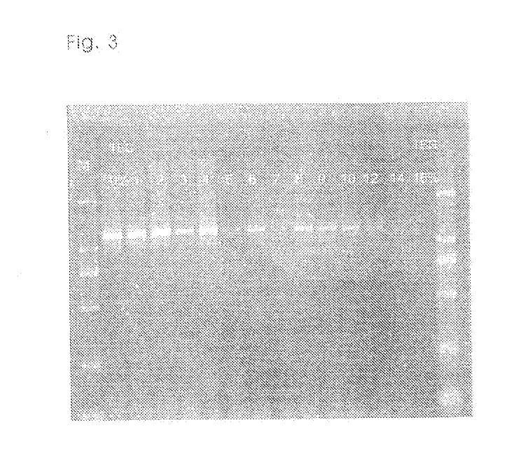

A PCR was carried out in the tumor suppressor gene Hugl (abbreviation for human giant larvae) of 3 ng purified human gDNA with the HotStarTaq® mastermix (25 μl final volume; 10 pmol forward primer (SEQ ID NO:4; 5′-CACACAGCGATGGCAGCTATGC-3′, 10 pmol reverse primer [SEQ ID NO:5; 5′-CCCAGTGATGGGCCAGCTC-3′) in the presence of rising concentrations of TEG in the reaction batch with the temperature profile 15 min 95° C., 40×[30 sec 95° C., 30 sec 59° C., 1 min 72° C.], 5 min 72° C. It is known that this reaction is very susceptible to interference with the primers used, e.g. with regard to contaminations of the DNA template. The PCR product yield in the reaction batches was then examined in conclusion by agarose gel electrophoresis. The result is shown in FIG. 3.

Result: The Hugl PCR carried out here can also be carried out sucessfully in the presence of large quantities of TEG (10 vol %).

Examples 1-3 demonstrate the superiority of TEG-containing washing buffers over ethanol-containing washing, buffers when using the MagAttract® technology for DNA purification. TEG washing buffers are particularly advantageous if large quantities of the produced DNA-containing eluate are to be used in enzyme-dependent amplification reactions (in this case quantitative PCR). This option is particularly important if the sample material used contains only minute nucleic acid quantities (viruses in low titers, single cells of forensic material, etc.).

The following examples demonstrate that TEG-containing washing buffers are unsuitable for RNA extraction from biological samples. Since TEG does not negatively affect the PCR reaction, as was demonstrated above, this observation is probably to be ascribed to a disruption of the activity of the reverse transcriptase by TEG.

### Example 4

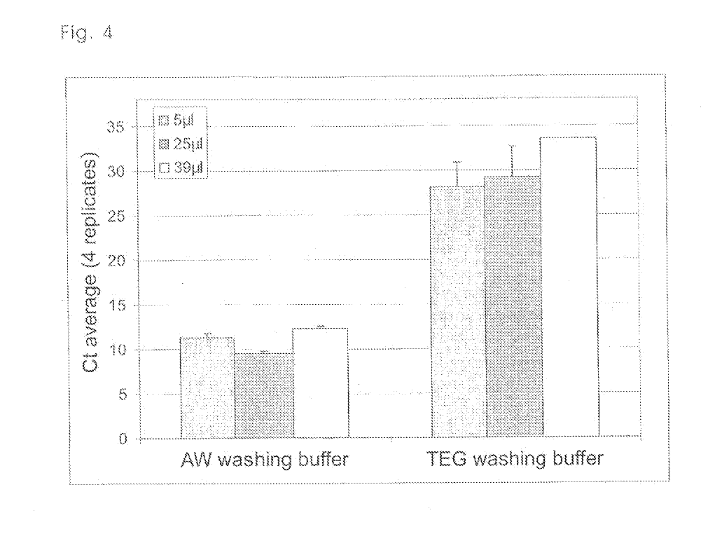

A joint lysate of a mixture of 30 μl bacteriophage fr (a bacteria-specific virus with RNA-based genome), 3 ml blood, 300 μl Qiagen proteinase K and 3 ml GIN3 was incubated for 15 min at 56° C. SPE (solid phase extraction) was carried out solely by means of the MagAttract® method. Similar to the description in Example 1, the large lysate batch was divided into 600 μl portions and in each case 300 μl isopropanol and 30 μl MasG® was added. For washing the bound nucleic acid, either AW1/AW2 or TEG1/TEG2 was used again. A washing process was carried out once with 1 ml AW1 or TEG1, respectively, and twice with 0.5 ml AW2 or TEG2, respectively. This was followed by 5 minutes of air drying. Finally, the purified RNA was eluted with 100 μl water. All SPE steps (binding/washing/eluting) were carried out using the robot BioSprint® 15 (Qiagen, Hilden). The RNA thus obtained was subsequently used in an RT-qPCR. For this purpose 5 μl, 25 μl or 39 μl eluate, respectively, was mixed with 10 μl QuantiTect® 5× Virus mastermix (Qiagen, Hilden), 20 pmol primers in each case (forward: SEQ ID NO:6; 5′-CTTCTGATCCGCATAGTGACGAC-3′; reverse: SEQ ID NO:7; 5′-AACGGTCATTCGCCTCCAGCAG-3′) and 10 pmol probe (SEQ ID NO:8; 6-FAM-TAGGGGATGGTAACGACGAAGCA-BHQ1) against the fr genome, and 0.5 μl QuantiTect Virus PT Mix (final volume 50 μl). The following temperature profile served for the cDNA production and amplification: 20 min 50° C., 5 min 95° C., 40×[15 sec 95° C., 45 sec 60° C.]. The result is shown in FIG. 4.

Result: Basically, the eluates washed with AW buffers show a significantly better RT-qPCR performance (tower Ct value) than the batches washed with the TEG buffers. Moreover, it is found that the use of large quantities of TEG eluate (39 ml to 50 μl RT-qPCR batch) not only leads to a poorer Ct value as compared to the use of smaller TEG eluate quantities (5 μl to 50 μl final volume), but may also lead to a total failure of the amplification reaction (only 1 of 4 batches with 39 ml per 50 μl reaction batch exhibited a successful amplification reaction).

One explanation of the poor performance of the tetraethylene glycol-washed samples is the fact that TEG depletion is not ensured due to the air drying process that was carried out. TEG has a boiling temperature of 306° C. and will therefore not transition into the gas phase during air drying.

### Example 5

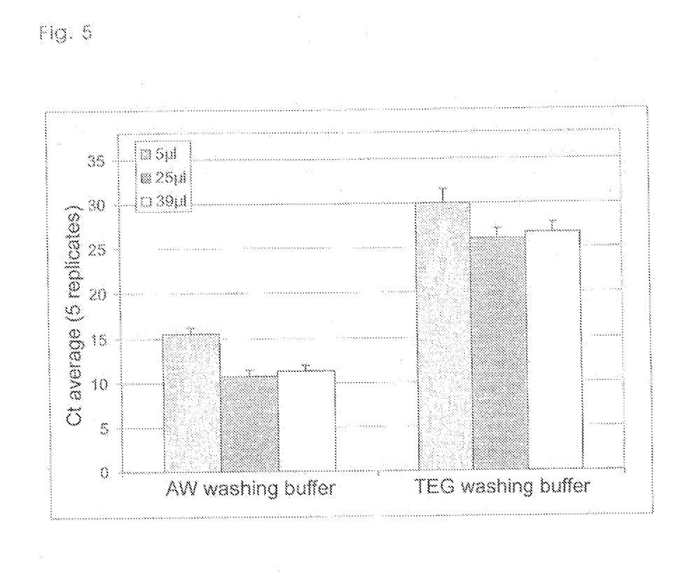

Bacteriophage fr was also added to human blood and lysed, and the RNA was purified also in this example. All lysis and SPE steps were carried out in a manner identical to example 4. However, in order to remove TEG remaining after the washing steps as effectively as possible, the air drying process was in this case replaced with a water rinsing process. Again, 5 μl, 25 μl and 39 μl, respectively, were mixed for the subsequent RT-qPCRs with the same PCR components as in Example 4 to result in a final volume of 50 μl. FIG. 5 shows the result.

Result: As in Example 4, the eluates washed with AW buffers show a significantly better RT-qPCR performance than the batches washed with the TEG buffers. This time, the inhibition while using large quantities of TEG eluate (39 μl per 50 μl RT-qPCR batch) is much lower. This shows that washing with water removes residual TEG from the Mag-Beads® better than drying in air. Nevertheless, it remained inexplicable up to this point why the TEG eluates obtained poorer results as a matter of principle than the AW eluates.

### Example 6

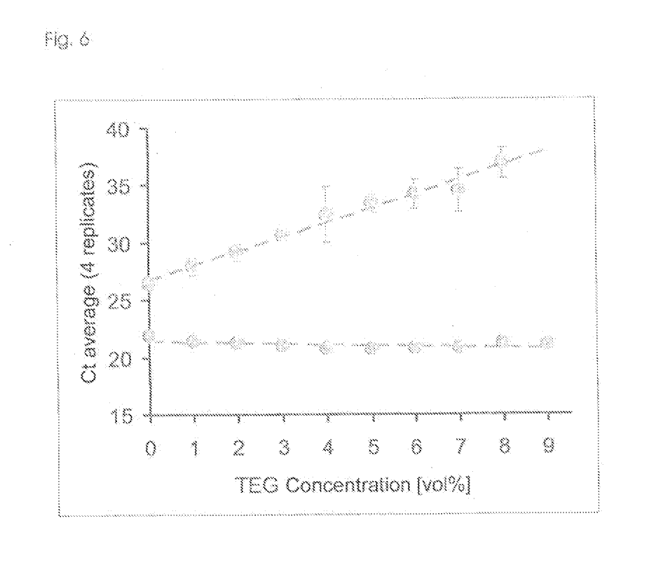

The question that remained in Example 5, namely why the TEG eluates yielded poorer RT-qPCR results than the AW eluates as a matter of principle was pursued in the following experiment, A RT-qPCR (QuantiTect® Probe RTPCR Mastermix) of purified RNA from bacteriophage fr and a qPCR (QuantiTect® Probe PCR Mastermix) of purified human gDNA (forward primer: SEQ ID NO:9; 5′-GCCGCTAGAGGTGAAATTCTTG-3′, reverse primer SEQ ID NO:10; 5′-CATTCTTGGCAAATTTCG-3′, probe: SEQ ID NO:11; HEX-ACCGGCGCAAGACGGACCAGA-BHQ1), respectively, was carried out in the presence of rising concentrations of TEG in the reaction batch (4 replicates each). The temperature profile used was 30 min 50° C., 15 min 95° C., 4×[15 sec 95° C., 1 min 60° C.]. The result is shown in FIG. 6.

Result: Even in larger concentrations, TEG does not change for the worse the performance of the PCR reaction in the case of DNA. This result confirms the data of the Hugl PCR presented in Example 3. In contrast, already small qunatities of TEG (from 1 vol %) lead to a dramatic deterioration of the RT-qPCR reaction efficiency in the case of RNA. At 9 vol % TEG in the batch, none of the 4 RT-qPCR reactions was successful (no amplification discernible).

1. A process for amplifying DNA, comprising amplifying DNS using a reaction solution comprising tetraethylene glycol.

2. The process according to claim 1, wherein the tetraethylene glycol in the reaction solution originates from eluate of a DNA preparation on a solid surface that was used for preparing the DNA.

3. A process for the preparation and amplification of DNA, comprising:
a) immobilizing DNA contained in a solution on a surface
b) washing the surface with a washing buffer
c) recovering the DNA by elution,
d) carrying out a DNA amplification with eluate obtained in step c),
wherein the washing buffer comprises tetraethylene glycol.

4. The process according to claim 3, wherein the tetraethylene glycol in the washing buffer is optionally at least 10 vol %.

5. The process according to claim 3, wherein the volume of the eluate in step d) is at least 10 vol %, optionally at least 30 vol % based on the entire reaction volume of the amplification.

6. The process according to claim 1, wherein the tetraethylene glycol in the entire reaction volume of the amplification is up to 20 vol %.

7. The process according to claim 3, wherein the washing buffer moreover contains one or more constituents selected from the group consisting of chaotropic salts and buffering substances.

8. The process according to claim 1, wherein the DNA amplification is carried out as an end-point PCR, quantitative PCR or isothermal amplification.

9. The process according to claim 1, wherein said process is carried out in a microfluidic device optionally comprising lab on a chip, LoC.

10. A kit of parts for DNA amplification and DNA preparation according to the process according claim 3, comprising:
a solution 1 as a washing buffer comprising tetraethylene glycol, and
one or more reagents required for carrying out the DNA amplification.

11. A kit of parts according to claim 10, comprising one or more of the following constituents, selected from
a solution 2, comprising a binding mediator,
a solution 3, comprising an elution agent,
a solution 4 comprising a lysis buffer and optionally one or more proteases;
a microfluidic device optionally comprising lab on a chip, LoC.

12. A kit of parts according to claim 10, which is capable of being used for preparation and amplification of DNA obtained from bilogical materials.

13. A kit of parts according to claim 10, which is capable of being used for preparation and amplification of DNA obtained from samples taken for molecular-diagnostic purposes optionally comprising human and/or veterinary medicine, and optionally from forensic samples and/or from viruses or bacteria contained in blood.

14. Use of Tetraethylene glycol used in a reaction solution for carrying out a DNA amplification.

15. The tetraethylene glycol according to claim 14, wherein the DNA amplification is an end-point PCR, quantitative PCR or isothermal amplification.

16. A reaction solution for carrying out a DNA amplification, comprising one or more reagents required for amplification and tetraethylene glycol.

17. The reaction solution according to claim 13, wherein the tetraethylene glycol in the reaction solution is up to 20 vol %.


SEQUENCE LISTING (11 sequences)

SEQ ID NO: 1 - DNA, 24 bp, Artificial Sequence
  Forward Primer
cgatgatgctacccctgaaaaact

SEQ ID NO: 2 - DNA, 25 bp, Artificial Sequence
  Reverse Primer
tattgtcgcttgaactgatttcctc

SEQ ID NO: 3 - DNA, 25 bp, Artificial Sequence
  Probe
cgttgttaagtcaatggaaaacctg

SEQ ID NO: 4 - DNA, 22 bp, Artificial Sequence
  Forward Primer
cacacagcgatggcagctatgc

SEQ ID NO: 5 - DNA, 19 bp, Artificial Sequence
  Reverse Primer
cccagtgatgggccagctc

SEQ ID NO: 6 - DNA, 22 bp, Artificial Sequence
  Forward Primer
cttctgatccgcatagtgacga

SEQ ID NO: 7 - DNA, 22 bp, Artificial Sequence
  Reverse Primer
aacggtcattcgcctccagcag

SEQ ID NO: 8 - DNA, 23 bp, Artificial Sequence
  Probe
taggggatggtaacgacgaagca

SEQ ID NO: 9 - DNA, 22 bp, Artificial Sequence
  Forward Primer
gccgctagaggtgaaattcttg

SEQ ID NO: 10 - DNA, 20 bp, Artificial Sequence
  Reverse Primer
cattcttgcaaatgctttcg

SEQ ID NO: 11 - DNA, 21 bp, Artificial Sequence
  Probe
accggcgcaagacggaccaga